(19) United States
(12) Patent Application Publication
(10) Pub. No.: US 2015/0265464 A1
(43) Pub. Date: Sep. 24, 2015

(54) LASER APPARATUS FOR TREATING TISSUE IN THE ANTERIOR PORTION OF AN EYE

(71) Applicant: Carl Zeiss Meditec AG, Jena (DE)

(72) Inventors: Christian Beder, Aalen (DE); Artur Hoegele, Oberkochen (DE); Christoph Hauger, Aalen (DE); Thorsten Tritschler, Aalen (DE)

(21) Appl. No.: 14/666,173

(22) Filed: Mar. 23, 2015

(30) Foreign Application Priority Data
Mar. 21, 2014 (DE) 10 2014 004 027.5

(51) Int. Cl. A61F 9/008

(52) U.S. Cl. CPC A61F 9/008; A61F 9/00814; A61F 9/00823; A61F 9/0084; A61F 2009/00897; A61F 2009/00863; A61F 2009/0087; A61F 2009/00872

(57) ABSTRACT

A laser apparatus for treating tissue in the anterior portion of an eye including a laser for generating a light beam, an optical device for shaping a light distribution and an imaging optical system for imaging the light distribution in a focal plane. The imaging optical system is embodied in such a way that a pupil of the imaging optical system is formed between 2 and 25 mm in front of or behind the focal plane.

### CROSS REFERENCE TO RELATED APPLICATION

This application claims priority of German patent application no. 10 2014 004 027.5, filed Mar. 21, 2014, the entire content of which is incorporated herein by reference.

### FIELD OF THE INVENTION

The invention relates to a laser apparatus for treating tissue in the anterior portion of an eye, including a laser for generating a light beam, an optical device for shaping a light distribution and an imaging optical system for imaging the light distribution in a focal plane.

### BACKGROUND OF THE INVENTION

EP 0 467 775 B1 has disclosed a laser apparatus for cutting a lens capsule, which has a device for generating an infrared pulsed laser beam and a device for projecting the laser beam onto the lens capsule so as to cut the latter. The projection device includes an optical focusing device for focusing the laser beam and an axicon lens device for projecting the focused beam onto the lens capsule in a ring-shaped form.

U.S. Pat. No. 8,562,596 B2 has disclosed a further laser apparatus for cutting a lens capsule. To this end, a punctiform laser beam is directed onto the eye and guided in a scanning method along a predetermined curve.

A disadvantage of the aforementioned laser apparatuses is that the focused laser beam is incident on the retina of the eye at, or in the vicinity of, the macula with a high power density. Hence, the region of the retina and, in particular, the macula, is exposed to a high load.

### SUMMARY OF THE INVENTION

It is an object of the invention to provide a laser apparatus, via which tissue in the anterior portion of an eye, in particular in a radial edge region of the anterior portion, can be treated, with other tissue layers of the eye being spared.

This object is achieved by a laser apparatus having a laser configured to generate a light beam, an optical arrangement configured to shape a light distribution, and an imaging optical system configured to image the light distribution in a focus plane. According to the invention, the imaging optical system is embodied in such a way that a pupil of the imaging optical system is formed between 2 and 25 mm in front of or behind the focal plane.

Prior to the treatment, a patient is placed in front of the laser apparatus in such a way that the tissue to be treated in the anterior portion is arranged in the focal plane of the laser apparatus. What should be noted here is that the focal plane of the laser apparatus is slightly shifted by the optically effective constituents of the anterior portion, in particular by the curved cornea. Imaging the laser beam in the focal plane, in which the tissue to be treated (for example, the capsular bag of the lens) is also arranged, brings about a high power density at the location of the focus.

A consequence of the embodiment of the laser apparatus in such a way that the pupil is arranged at a distance of between 2 and 25 mm from the focal plane is that the light beam passes through the focal plane at an angle to the optical axis of the imaging optical system. What this achieves is that the laser beam is incident on the retina away from the macula, which is the most sensitive part of the retina. This ensures that the tissue treatment at the anterior portion is effected in such a way that both the other tissue layers in the anterior portion and the retina are spared to the greatest possible extent. Here, the aforementioned angle of the light beam in relation to the optical axis is defined as the angle which an angle bisector of the marginal rays of the light beam assumes in relation to the optical axis.

In one embodiment of the invention, the optical device for shaping a light distribution is configured in such a way that the light beam is convertible into a light beam with a line-shaped cross section. In this embodiment, a line focus is generable in the focal plane. As a result, it is possible to treat eye tissue in one work step (that is, without performing a scan) along the line determined by the line focus. In this way, it is possible to generate, for example, a tear or a cut in the tissue. Within the meaning of the present patent application, the term “line-shaped cross section” is understood to mean any line-shaped, straight or curved, closed or open, continuous or interrupted structures in general, the dimensions of which in the line direction are many times (for example, ten times, one hundred times or one thousand times) greater than across the line direction and which are imaged in the focal plane via the imaging optical system.

In a further embodiment of the invention, the optical device for shaping a light distribution includes a convex or concave axicon, a diffractive optical element, a reflection echelon grating and/or a micro-mirror array. Axicons, diffractive optical elements and reflection echelon gratings are members of a group of optical elements, via which light beams from lasers can easily be reshaped into light rays with ring-shaped round, oval or elliptical cross sections. Micro-mirror arrays, also known as “digital micro-mirror devices” (DMD), are constructed from many small switchable mirrors. With the aid thereof, it is possible to reshape a light beam from lasers into light rays with virtually any cross section.

In a further embodiment of the invention, the imaging optical system and the optical device for shaping a light distribution are configured in such a way that the light beam passes through the focal plane as a closed or interrupted ring structure with a radius of between 1.5 and 5 mm. In this embodiment, the laser apparatus is particularly suitable for performing a capsulotomy, that is, for opening the capsular bag.

In a further embodiment of the invention, the optical device for shaping a light distribution is embodied in a manner movable along an optical axis. As a result, the imaging scale, with which the laser beam is imaged in the focal plane, is variable. Furthermore, this allows a diameter of a line focus to be set in the case of a circular, oval or elliptic embodiment of the light beam.

In a further embodiment of the invention, the laser is embodied to emit a narrow-band light beam within a wavelength range from 525 nm to 675 nm, in particular from 550 nm to 610 nm, more particularly from 580 nm to 610 nm. This embodiment enables the tissue treatment to be performed in a particularly sparing manner. To this end, the tissue to be treated is enriched with a dye in a form bound to an extracellular matrix of the tissue or in a free form, the absorption maximum of which dye lies in the emission spectrum of the laser. When the enriched tissue is irradiated by the laser beam, there is an absorption superelevation in the tissue, via which the enriched, irradiated tissue is strongly heated locally, without the adjacent tissue parts being excessively damaged. The aforementioned emission wavelengths are adapted to use of trypan blue as a dye.

In a further embodiment of the invention, the imaging optical system includes a termination element with positive refractive power as the last optically effective element upstream of the focal plane. No further optically effective element, that is, no element with collecting or dispersing optical effect, is arranged between the termination element and the focal plane. By use of the termination element upstream of the focal plane, it is possible to adapt the location of the pupil plane relative to the focal plane in such a way that the tissue adjacent to the focal plane and the retina of the eye are exposed to a further reduced power density of the laser beam. Here, an aperture angle of the light beam and/or an irradiation angle of the light rays of the light beam on the focal plane are further increased.

In a further embodiment of the invention, the termination element is configured and arranged in the imaging optical system in such a way that an intermediate image of the focal plane is formed. As a result, it is possible to construct the imaging optical system from optical elements with smaller diameters such that, overall, a more compact overall system emerges.

It is a further object of the invention to provide an ophthalmic device having a surgical microscope and a laser apparatus for treating tissue in the anterior portion of an eye, while sparing the retina and adjacent tissue layers. This object is achieved by an ophthalmic device having a surgical microscope and a laser apparatus having a laser configured to generate a light beam, an optical arrangement configured to shape a light distribution and an imaging optical system configured to image the light distribution in a focus plane; and, the imaging optical system being configured so as to form a pupil of the imaging optical system between 2 mm and 25 mm ahead of or behind the focus plane.

In one embodiment of the invention, a coupling element for coupling the laser beam into the observation beam path is arranged in an observation beam path of the surgical microscope, wherein the termination element is arranged in the observation beam path. Here, the term “coupling” should be understood to mean that the optical axis of the imaging optical system of the laser apparatus corresponds to the optical axis of the observation beam path between the coupling element and the focal plane. In this case, the termination element is penetrated both by the laser beam and by the observation beam path of the surgical microscope. Particularly preferably, the surgical microscope is likewise focusable onto the focal plane of the laser apparatus so that processes in the focal plane can be observed directly through the surgical microscope.

In a further embodiment of the invention, the termination element has a first region, in which the positive refractive power is formed, and the termination element has a second region, which has a refractive power that differs from the positive refractive power formed in the first region, wherein the first region is penetrated by the light beam of the laser apparatus and the second region is penetrated by the observation beam path of the surgical microscope. As a result, it is possible to influence the observation beam path and the beam path from the laser apparatus independently of one another.

In a further embodiment, the second region is embodied as a plane parallel plate. This prevents the termination element from generating an intermediate image in the observation beam path which would otherwise lead to an image inversion further along the observation beam path, which would need to be compensated by a further inverter element in the observation beam path. Thus, a plane parallel plate in the second region of the termination element directly enables an image-side and non-reversed observation of the object by the surgical microscope without use of inverter elements.

### BRIEF DESCRIPTION OF THE DRAWINGS

The invention will now be described with reference to the drawings wherein:

FIG. 1 shows a first embodiment of a laser apparatus according to the invention in a first configuration;

FIG. 2 shows the beam path of the laser apparatus from FIG. 1 at the eye;

FIG. 3 shows, in an exemplary manner, the laser apparatus from FIG. 1 in a second configuration;

FIG. 4 shows the beam path of the laser apparatus from FIG. 3 at the eye;

FIG. 5 shows a second embodiment of the laser apparatus according to the invention;

FIG. 6 shows the beam path of the laser apparatus from FIG. 5 at the eye;

FIG. 7 shows a third embodiment of the laser apparatus according to the invention;

FIG. 8 shows the beam path of the laser apparatus from FIG. 7 at the eye;

FIG. 9 shows the laser apparatus from FIG. 1 in combination with a surgical microscope;

FIG. 10 shows a fourth embodiment of the laser apparatus according to the invention interacting with a surgical microscope;

FIG. 11 shows the laser apparatus from FIG. 5 in combination with a surgical microscope;

FIG. 12 shows the laser apparatus from FIG. 7 in combination with a surgical microscope;

FIG. 13 shows, in an exemplary manner, the laser apparatus from FIG. 7 in a variant integrated into a surgical microscope;

FIG. 14A shows a surgical microscope with a termination element in the observation beam path;

FIG. 14B shows a magnified illustration of part of the observation beam path of the surgical microscope through the termination element;

FIG. 15A shows a laser apparatus with the termination element from FIG. 14A in the laser beam; and,

FIG. 15B shows a magnified illustration of part of the laser beam through the termination element.

### DESCRIPTION OF THE PREFERRED EMBODIMENTS OF THE INVENTION

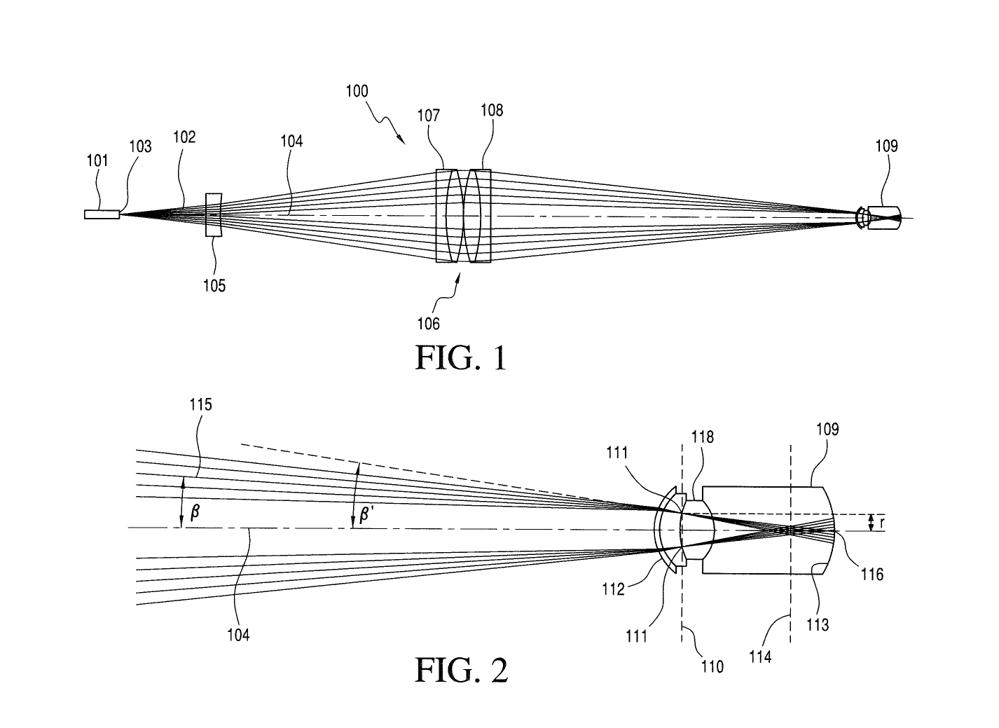

FIG. 1 depicts a first embodiment of a laser apparatus 100 according to the invention. The laser apparatus includes a laser 101 with an emergence surface 103, at which a narrow-band laser beam 102 with a compact cross section is emitted. The cross section of the laser beam 102 after emergence from the laser 101 can, for example, have an approximately round, rectangular or oval embodiment.

The laser apparatus 100 furthermore includes an optical device for shaping a light distribution in the form of an axicon 105 with a concave embodiment. The optical axis 104 of the axicon is arranged in the laser beam 102. The concave axicon 105 causes a deflection of the laser beam 102 away from the optical axis 104 and a reshaping of the light distribution of the laser beam 102. In the present embodiment, a round cross section of the laser beam 102 upon entry into the axicon 105 is assumed in the following text, which cross section is reshaped into a ring-shaped cross section by the axicon.

The laser beam with the ring-shaped cross section subsequently passes through an optical convergence element 106, which is formed from two cemented elements (107, 108) with positive refractive power in this embodiment.

The laser apparatus according to the invention is configured for treating tissue in the anterior portion of an eye. This is explained in more detail below on the basis of a capsulotomy, that is, an opening of the anterior lens capsular bag via a laser, on one eye 109.

As a result of the convergence element 106, an imaging optical system is formed, the system being arranged on a common optical axis 104 with the axicon 105. The imaging optical system is configured in such a way that a ring-shaped line focus 111 is formed in a focal plane 110.

For the purposes of performing the capsulotomy, a patient is placed in front of the laser apparatus 100 in such a way that the capsular bag 118 is arranged in the focal plane of the laser apparatus, wherein the line focus 111 defined by the axicon 105 corresponds to the desired cut line in the capsular bag. It should be noted here that the focal plane 110 of the laser apparatus is slightly displaced by the optical effect of the curved cornea 112 that was introduced into the beam path. In a subsequent step, the capsular bag is exposed to laser radiation for a short period of time of, for example, between 200 ms and 500 ms, as a result of which the capsular bag in the line focus is locally heated and thus it is severed or scored. As a result of the special shaping of the light beam, it is possible to perform the tissue treatment in one work step (and not sequentially like in a scanning method).

FIG. 2 depicts the beam path in the region of the eye in a magnified manner. In this embodiment, the ring-shaped line focus 111 has a radius (r) in the region of 1.5 to 5 mm in the focal plane 110.

Here, the imaging optical system is configured in such a way that a pupil in a pupil plane 114 of the imaging optical system is embodied between 2 and 25 mm behind the focal plane 110. Here, a pupil plane should be understood to mean a plane across the optical axis, in which a chief ray intersects the optical axis of the imaging optical system. What this achieves is that the light beam passes through the cornea 112 in the anterior portion of the eye and through the rear side of the capsular bag over a substantially larger cross-sectional area than the cross-sectional area of the line focus at the treatment location (the anterior side of the capsular bag). Furthermore, what the distance between the pupil plane and the focal plane achieves is that the light beam also illuminates a significantly larger cross-sectional area on the retina 113 of the eye than at the treatment location. As a result, the power density with which the laser beam passes through adjacent tissue layers or with which it is incident on the retina 113 is significantly lower than when it passes through the line focus 111 on the anterior side of the capsular bag.

Here, the light beam is incident on the cornea of the eye at an angle β (in relation to the optical axis 104) of between 5 and 58° and incident on the capsular bag at an angle β′. Here, the “angle of the light beam” should be understood to mean the angle between an angle bisector 115 and the optical axis 104. Here, as depicted in FIG. 2, the angle bisector is set in a plane which intersects the light beam and contains the optical axis 104, and it is defined as the angle bisector of the outer marginal rays of the light beam in this plane. The arrangement of the pupil location at a distance from the focal plane and the relatively large passage angle of the light beam through the focal plane connected therewith results in the light beam being incident on the retina away from the center 116 of the retina, as a result of which the risk of damage to the retina is reduced.

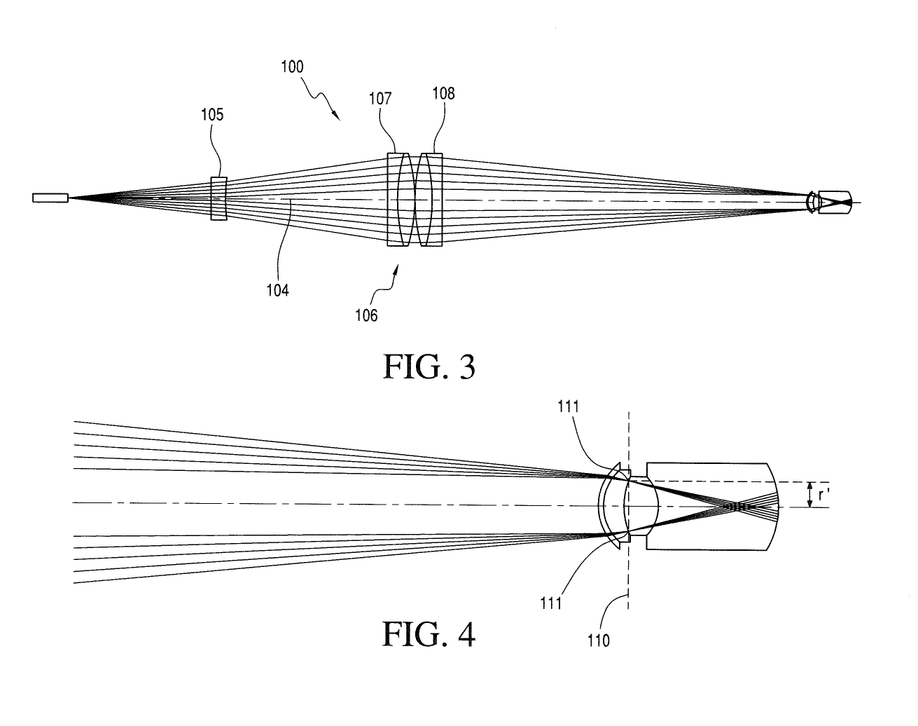

In FIG. 3, the laser apparatus 100 from FIG. 1 is depicted in another configuration, in which the axicon 105 is displaced along the optical axis 104 in the direction of the convergence element 106. In this manner, a radius r′ of the line focus 111 is variable in the focal plane 106, as is shown in greater detail in FIG. 4.

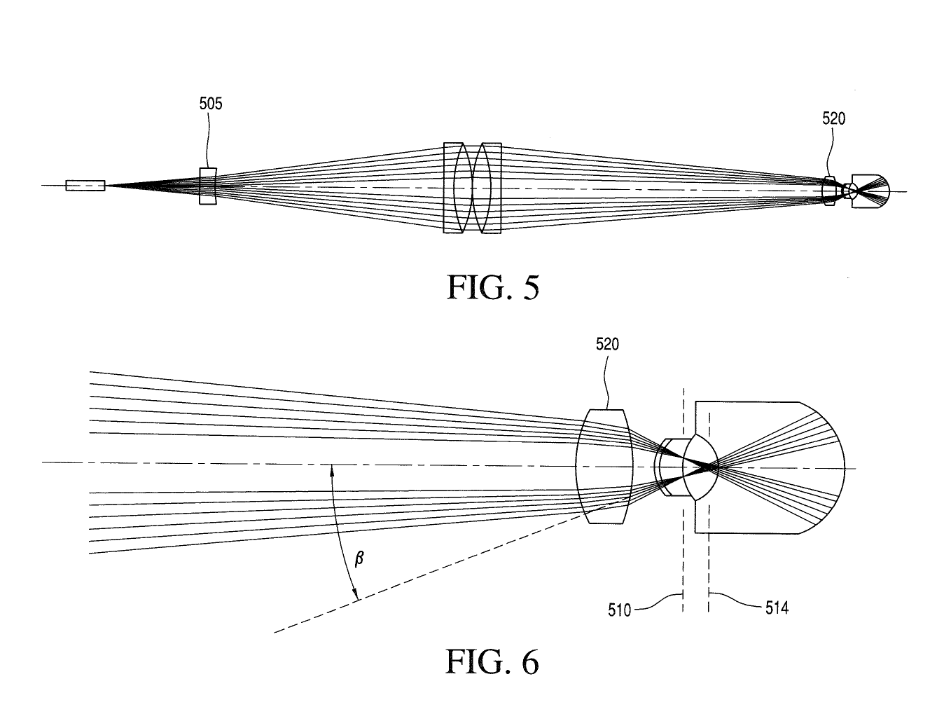

FIG. 5 depicts a second embodiment of a laser apparatus, which differs from the first embodiment in accordance with FIG. 1 in that the imaging optical system includes a termination element in the form of a single-lens element 520 with positive refractive power. In other embodiments, the termination element can also include a plurality of optical elements.

The single-lens element 520 brings about a deflection of the ring-shaped light beam in the direction of the optical axis, resulting in the distance between the pupil plane 514 and the focal plane 510 being significantly shorter and the light beam being incident on the cornea of the eye at a significantly larger angle β compared to the embodiment in accordance with FIG. 1. Consequently, the light rays of the light beam are incident on the retina at a significantly greater distance from the center 516 of the retina, as a result of which the exposure of the retina is further reduced. The refractive power of the single-lens element can be between 20 and 250 diopters.

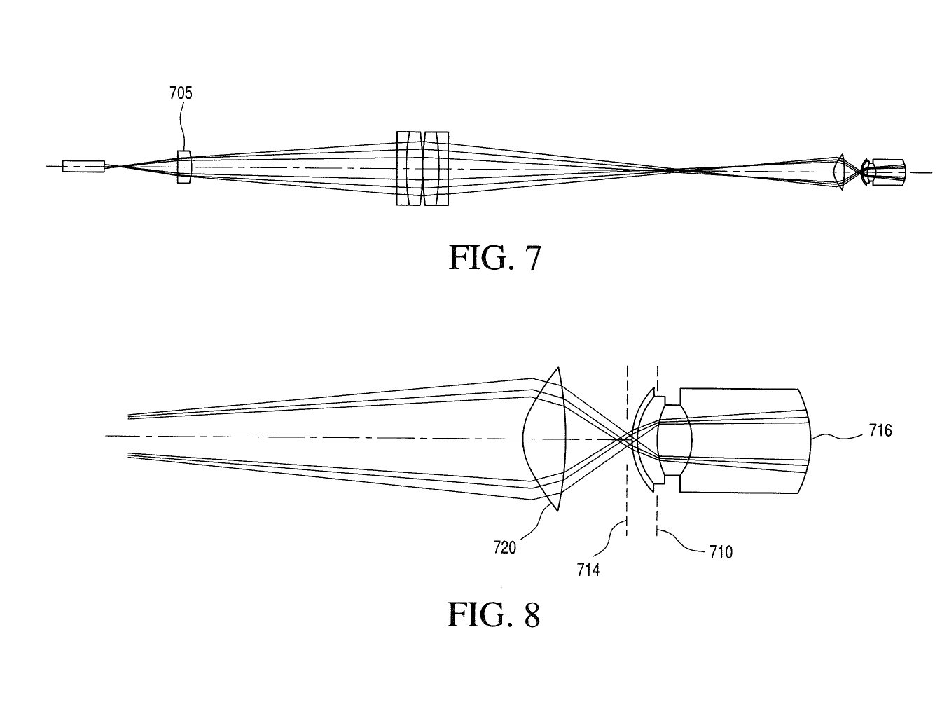

FIG. 7 and FIG. 8 show a third embodiment of the laser apparatus, which differs from the preceding embodiments in that an axicon 705 with a convex form is used instead of a concave axicon. As a result, a pupil is formed in a pupil plane 714 which, however, in contrast to the preceding embodiments, is arranged between 2 and 50 mm, in particular between 2 and 25 mm, in front of the focal plane 710. This embodiment also ensures that the light beam is incident on the retina at a distance from the center 716 of the retina and with a lower power density compared to the focal plane.

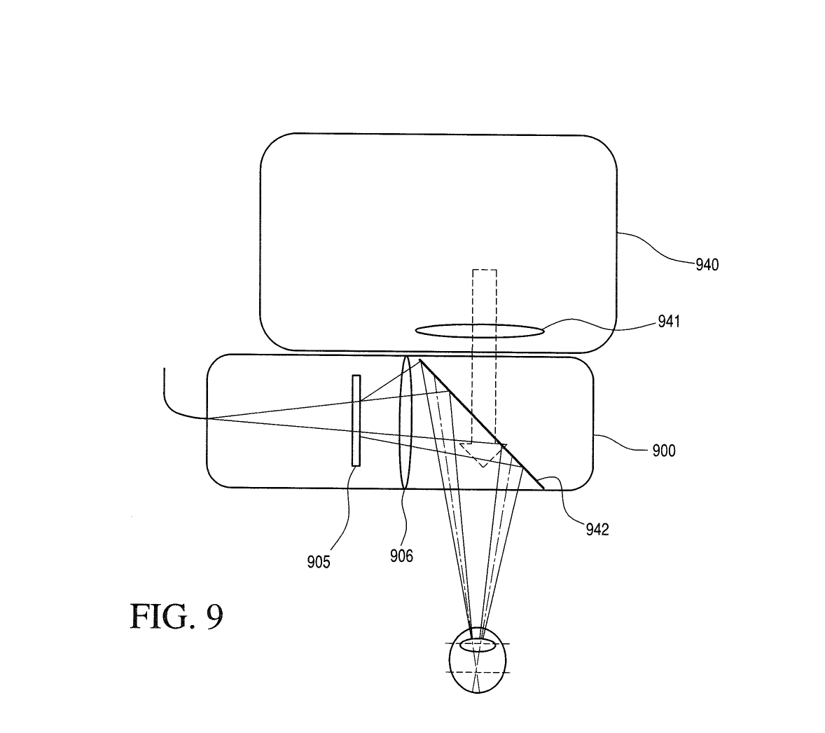

FIG. 9 depicts, in a schematic illustration, a laser apparatus 900 interacting with a surgical microscope 940, wherein, for simplification purposes, all that is shown of the surgical microscope 940 is a main objective 941 as an optical element. Analogous to the embodiment in accordance with FIG. 1, the laser apparatus 900 is equipped with a concave axicon 905 and a convergence element 906. Additionally, the laser apparatus 900 contains a coupling element 942, which is arranged in an observation beam path of the surgical microscope 940 in such a way that the optical axis of the laser apparatus at least largely corresponds to the optical axis of the observation beam path after the light beam was reflected at the coupling element. The coupling element 942 is preferably embodied as a dichroic mirror or beam splitter such that the laser light is reflected as completely as possible and the observation light is transmitted as completely as possible. As a result of combining the laser apparatus with a surgical microscope, it is possible to observe the treatment location prior to, during and after the treatment by means of the surgical microscope and monitor the progress of the treatment.

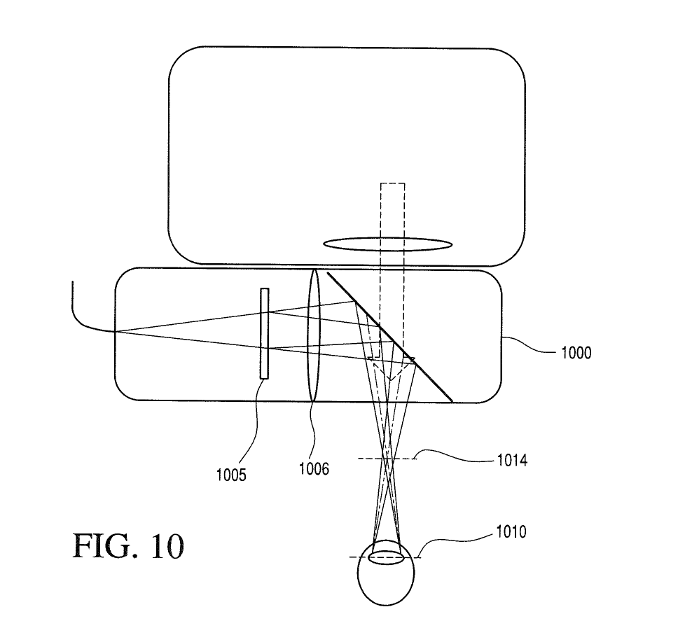

In the combination of laser apparatus 1000 and surgical microscope 1040 shown in FIG. 10, the laser apparatus 1000 differs from the preceding laser apparatuses in that an axicon 1005 in convex form is used as a beam deflection element, like in the third embodiment, but use is not made of a single-lens element as a termination element in the beam path. The pupil plane 1014 is once again embodied in front of the focal plane 1010, that is, between the focal plane 1010 and the convergence element 1006. In this embodiment, the selected parameter combination also ensures that the light beam is incident on the retina at a distance from the center of the retina and with a low power density.

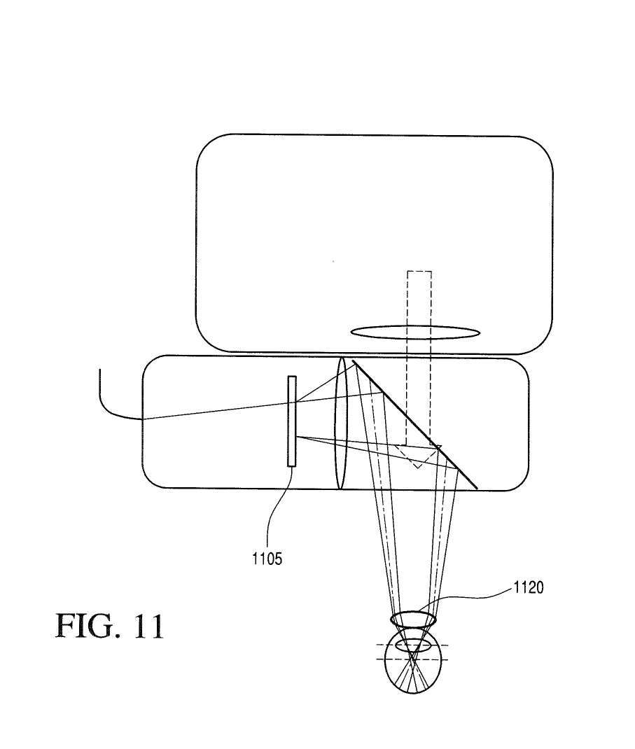

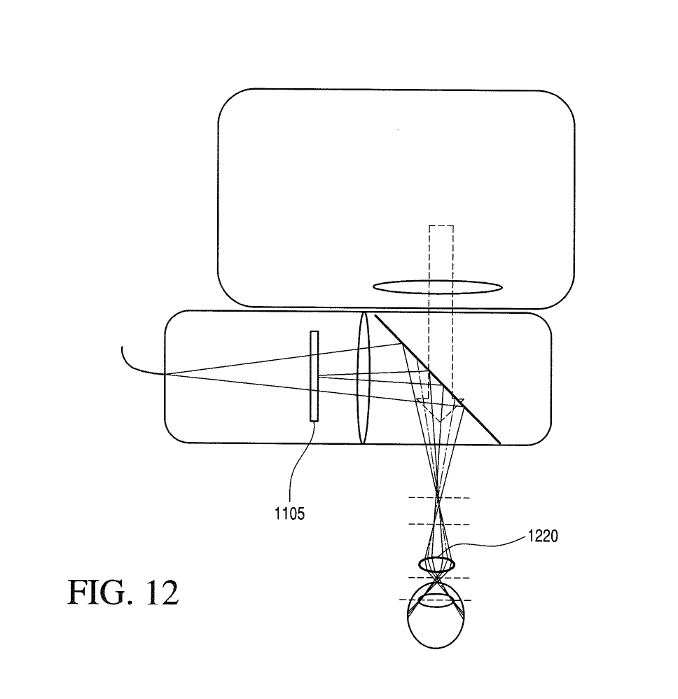

Similar combinations of laser apparatus and surgical microscope are also possible for laser apparatuses which, analogously to the embodiment in accordance with FIG. 5, are equipped with a concave axicon (505, 1105) as a beam deflection element and a single-lens element (520, 1120) as termination element, see FIG. 11. It is likewise possible to combine a laser apparatus in accordance with FIG. 7 with a surgical microscope, which laser apparatus is equipped with a convex axicon (705, 1205) and a single-lens element (720, 1220) as a termination element with positive refractive power, see FIG. 12.

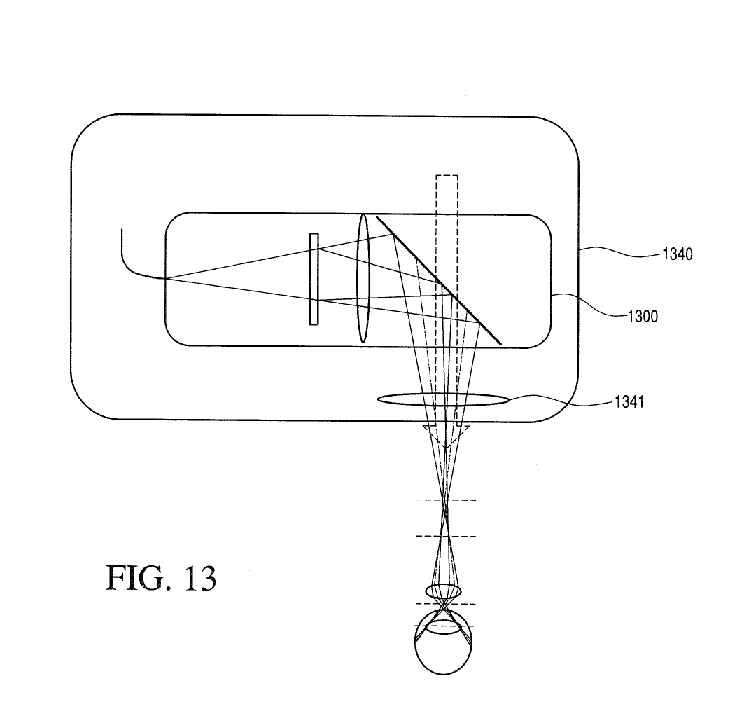

In further embodiments, depicted in an exemplary manner on the basis of FIG. 13 for a laser device with a concave axicon and a single-lens element as a termination element, the laser apparatus 1300 is integrated into the surgical microscope 1340 in such a way that the main objective 1341 of the surgical microscope is arranged in the laser beam.

The combinations of laser apparatuses and surgical microscopes depicted in FIGS. 11, 12 and 13 are disadvantageous in that the image of the object plane generated in the surgical microscope is inverted due to the single-lens element arranged in the beam path. In order to compensate for this inversion, further inversion optics, for example, in the form of an inverter tube, can be provided at a suitable location in the beam path of the surgical microscope.

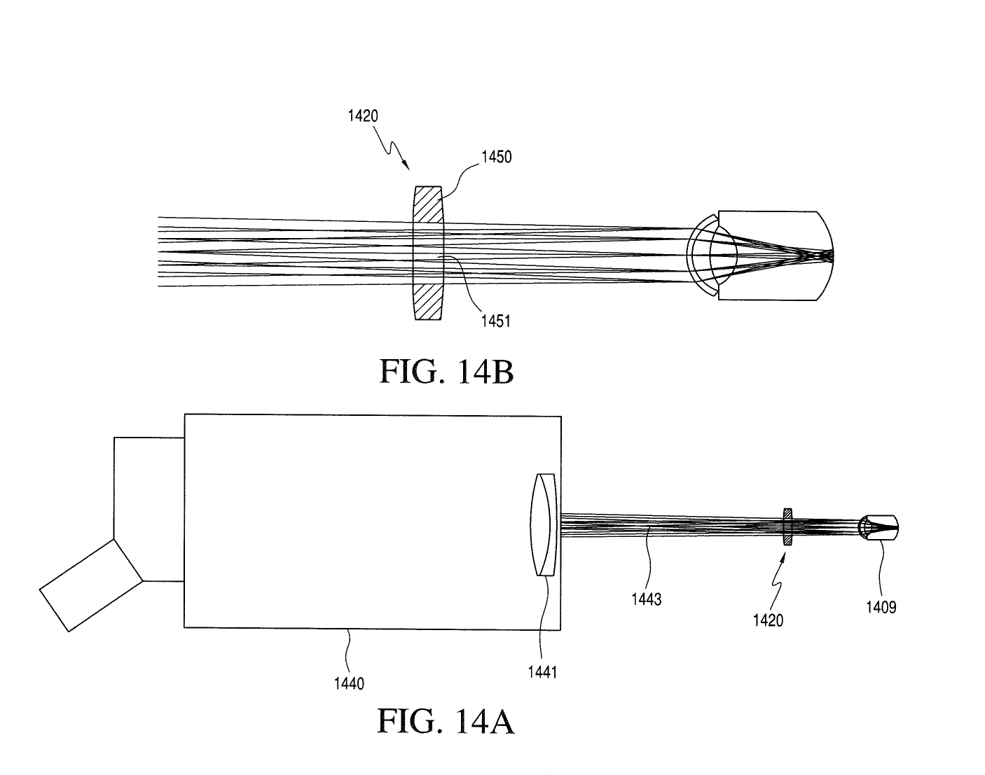

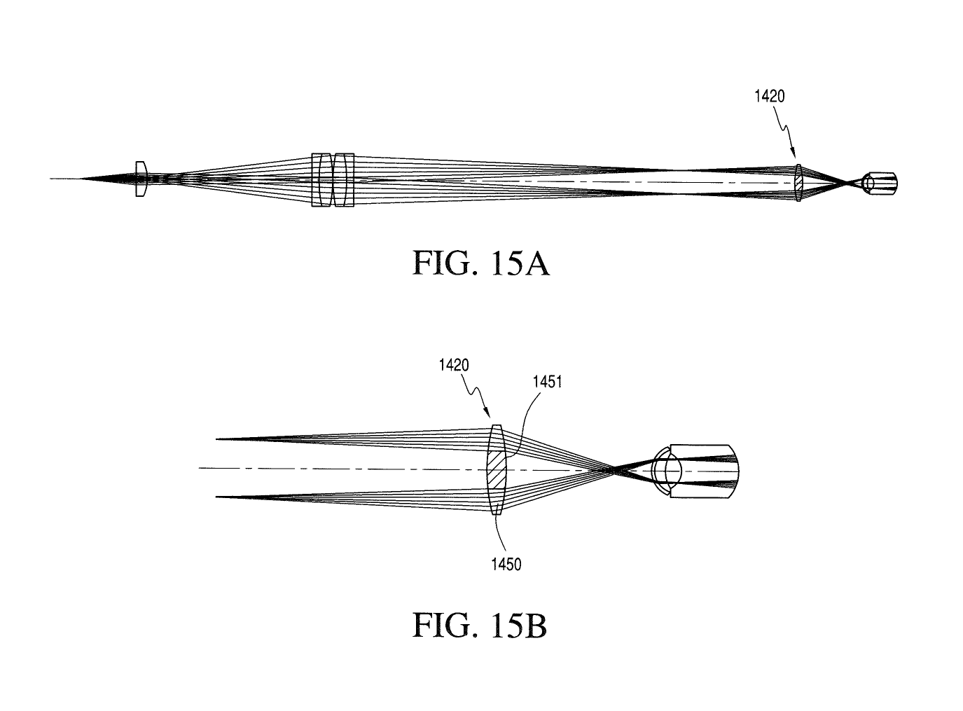

An alternative combination of laser apparatus and surgical microscope including a single-lens element in the laser beam path, which makes do without inversion optics in the surgical microscope, is presented below on the basis of FIGS. 14A, 14B, 15A and 15B.

FIG. 14A shows a surgical microscope 1440 including a main objective 1441, and the observation beam path 1443 of the surgical microscope between the main objective and the eye 1409 to be observed. A termination element 1420 of a laser apparatus is arranged in the observation beam path. The beam path associated with the laser apparatus can be gathered from FIG. 15A.

FIG. 14B depicts the profile of the observation beam path through the termination element 1420 in more detail. The termination element 1420 is subdivided into two regions with different refractive powers, wherein the second region 1451 is arranged around the optical axis of the termination element 1420 and the first region 1450 radially encloses the second region 1451.

In the second region 1451, the termination element 1420 is configured as a plane parallel plate such that a light ray does not experience a change in direction when passing through the plane parallel plate.

The termination element is embodied and arranged in the beam path in such a way that the observation beam path of the surgical microscope is guided through the second region 1451, which is embodied as a plane parallel plate. As a result, there is no image inversion in the observation beam path, and so further inversion optics can be dispensed with.

FIG. 15B depicts the profile of the beam path in the laser beam through the first region 1450 of the termination element in greater detail. Like in the embodiments described above, the first region is configured as a refractive, diffractive or reflecting element.

In further embodiments, diffractive optical elements, reflection echelon gratings and/or micro-mirror arrays are used in place of the described concave and convex axicons. Micro-mirror arrays, in particular, are distinguished by the fact that they can be used to generate almost any beam cross section such that a multiplicity of different tissue cuts are performable with laser apparatuses equipped accordingly.

It is understood that the foregoing description is that of the preferred embodiments of the invention and that various changes and modifications may be made thereto without departing from the spirit and scope of the invention as defined in the appended claims.

What is claimed is:

1. A laser apparatus for treating tissue in the anterior portion of an eye, the laser apparatus comprising:
a laser configured to generate a light beam;
an optical arrangement configured to shape a light distribution;
an imaging optical system configured to image said light distribution in a focus plane; and,
said imaging optical system being configured so as to form a pupil of said imaging optical system between 2 mm and 25 mm ahead of or behind said focus plane.

2. The laser apparatus of claim 1, wherein said optical arrangement is configured to transition said light beam into a light beam having a line-shaped cross-section.

3. The laser apparatus of claim 1, wherein said optical arrangement includes at least one of a convex axicon, a concave axicon, a diffractive optical element and a micro-mirror array.

4. The laser apparatus of claim 1, wherein:
said imaging optical system and said optical arrangement are configured so as to cause said light beam to pass through said focus plane as a closed or interrupted annular structure having a radius lying in a range between 1.5 mm and 5 mm.

5. The laser apparatus of claim 1, wherein said optical arrangement defines an optical axis and said optical arrangement is movable along said optical axis.

6. The laser apparatus of claim 1, wherein said laser is configured to emit a narrow band light beam in a wavelength range of 525 nm to 675 nm.

7. The laser apparatus of claim 1, wherein:
said imaging optical system includes a termination element having a positive refractive power; and,
said termination element is the last optically effective element upstream of said focus plane.

8. The laser apparatus of claim 7, wherein said termination element is configured and arranged in said imaging optical system so as to form an intermediate image of said focus plane.

9. An ophthalmic device comprising:
a surgical microscope;
a laser apparatus having a laser configured to generate a light beam, an optical arrangement configured to shape a light distribution and an imaging optical system configured to image said light distribution in a focus plane; and,
said imaging optical system being configured so as to form a pupil of said imaging optical system between 2 mm and 25 mm ahead of or behind said focus plane.

10. The ophthalmic device of claim 9 further comprising:
a coupling element;
said imaging optical system including a termination element having a positive refractive power;
said termination element being the last optically effective element of said laser apparatus upstream of said focus plane;
said surgical microscope defining a viewing beam path;
said coupling element being arranged in said viewing beam path and configured to couple said light beam into said viewing beam path; and,
said termination element being arranged in said viewing beam path.

11. The ophthalmic device of claim 10, wherein:
said positive refractive power is a first refractive power;
said termination element includes a first region having said first refractive power embodied therein;
said termination element further includes a second region having a second refractive power which is different than said first refractive power;
said first region is configured to have said light beam of said laser apparatus pass therethrough; and,
said second region is configured to have said viewing beam path of said surgical microscope pass therethrough.

12. The ophthalmic device of claim 11, wherein said second region is configured as a plane parallel plate.
